(19) United States
(12) Patent Application Publication
(10) Pub. No.: US 2020/0084344 A1
(43) Pub. Date: Mar. 12, 2020

(54) CAMERA HEAD

(71) Applicant: Sony Olympus Medical Solutions Inc., Tokyo (JP)

(72) Inventor: Atsuomi OHNO, Tokyo (JP)

(73) Assignee: Sony Olympus Medical Solutions Inc., Tokyo (JP)

(21) Appl. No.: 16/556,250

(22) Filed: Aug. 30, 2019

(30) Foreign Application Priority Data
Sep. 6, 2018 (JP) 2018-167249

(51) Int. Cl. H04N 5/225; A61B 1/00; A61B 1/06; A61B 1/04

(52) U.S. Cl. CPC H04N 5/2251; A61B 1/00066; A61B 1/00195; H04N 2005/2255; A61B 1/042; A61B 1/00105; A61B 1/0661

(57) ABSTRACT

A camera head includes a coupler member which is detachably connected to an eyepiece unit of an endoscope and configured to capture an object image emitted from the eyepiece unit. The coupler member includes an abutting portion which is rigid and configured to abut on the eyepiece unit at a plurality of points when at least the eyepiece unit is coupled, and a biasing member configured to apply a biasing force for the eyepiece unit to the abutting portion.

### CROSS-REFERENCE TO RELATED APPLICATION

The present application claims priority to and incorporates by reference the entire contents of Japanese Patent Application No. 2018-167249 filed in Japan on Sep. 6, 2018.

### BACKGROUND

The present disclosure relates to a camera head.

In the related art, an endoscope apparatus that observes the inside of a subject such as a person or a mechanical structural body is known in the medical field and the industrial field (for example, see JP H10-295638 A).

The endoscope apparatus disclosed in JP H10-295638 A includes an endoscope that captures an object image in a subject and emits the object image from an eyepiece unit, and a camera head that includes a coupler member to which the eyepiece unit is detachably connected and captures the object image emitted from the eyepiece unit.

The coupler member has a coupling recess portion into which the eyepiece unit is inserted. Thus, in a state where the eyepiece unit is inserted into the coupling recess portion, and the eyepiece unit is coupled to the coupler member, the endoscope and the camera head turn into a state of being are relatively rotatable around the central axis of the endoscope in an insertion direction into the subject.

### SUMMARY

Generally, a locking member that locks the eyepiece unit to prevent removal of the eyepiece unit is provided in the coupling recess portion. The locking member is formed, for example, with an elastic material such as rubber. However, the locking member formed with an elastic material such as rubber has a problem that, if rotational torque increases when the locking member relatively rotates the endoscope and the camera head around the central axis of the endoscope, frictional resistance between the locking member and the eyepiece unit increases, and thus the degree of wear of the locking member also increases.

There is a need for a camera head in which it is possible to suppress wear of the locking member to lock the endoscope, due to rotation of the endoscope.

According to one aspect of the present disclosure, there is provided a camera head including: a coupler member which is detachably connected to an eyepiece unit of an endoscope and configured to capture an object image emitted from the eyepiece unit, wherein the coupler member includes an abutting portion which is rigid and configured to abut on the eyepiece unit at a plurality of points when at least the eyepiece unit is coupled, and a biasing member configured to apply a biasing force for the eyepiece unit to the abutting portion.

### BRIEF DESCRIPTION OF THE DRAWINGS

FIG. 1 is a diagram illustrating an overall configuration of an endoscope apparatus according to an embodiment;

FIG. 2 is a diagram illustrating a connection portion between an endoscope and a camera head;

FIG. 5 is a diagram illustrating a configuration of a coupler member in the camera head when viewed from an arrow A illustrated in FIG. 2;

FIG. 4 is a diagram illustrating the connection portion between the endoscope and the camera head;

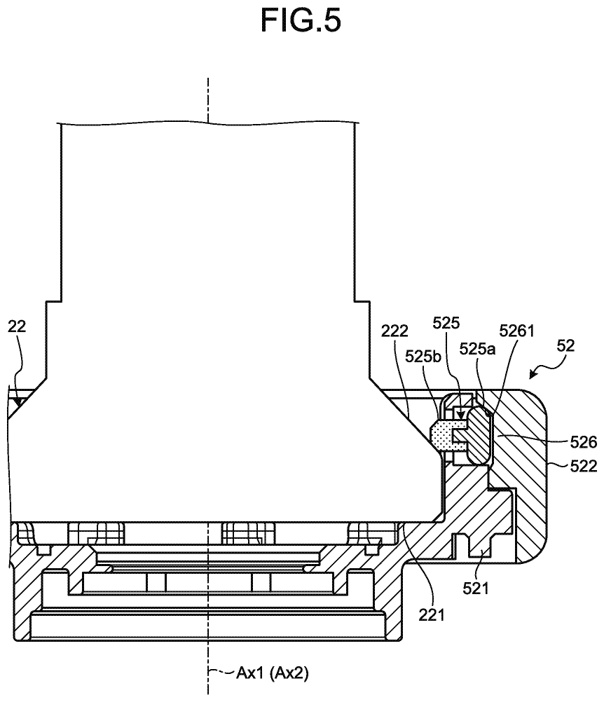

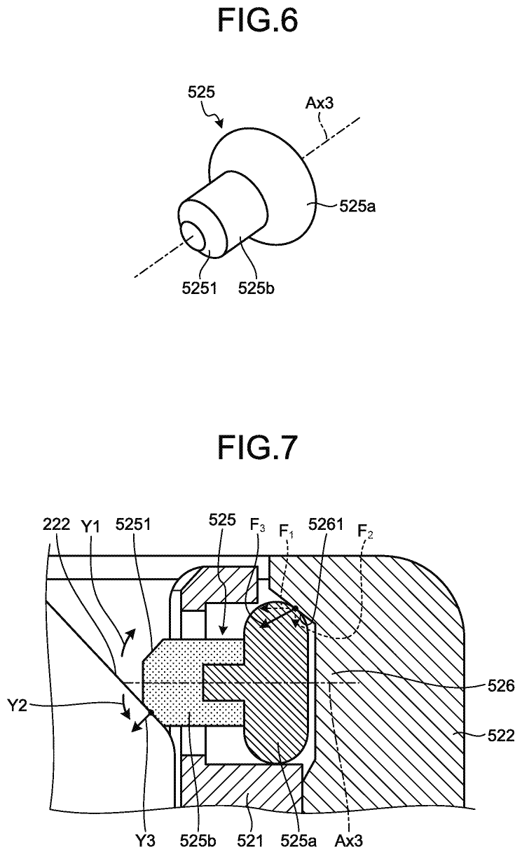

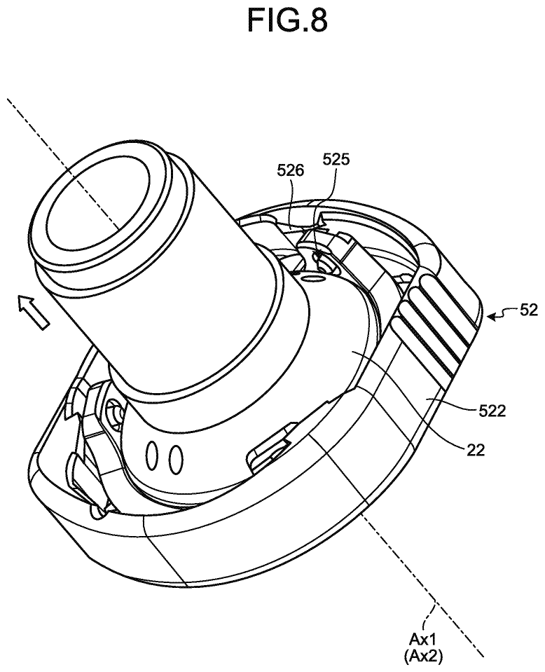

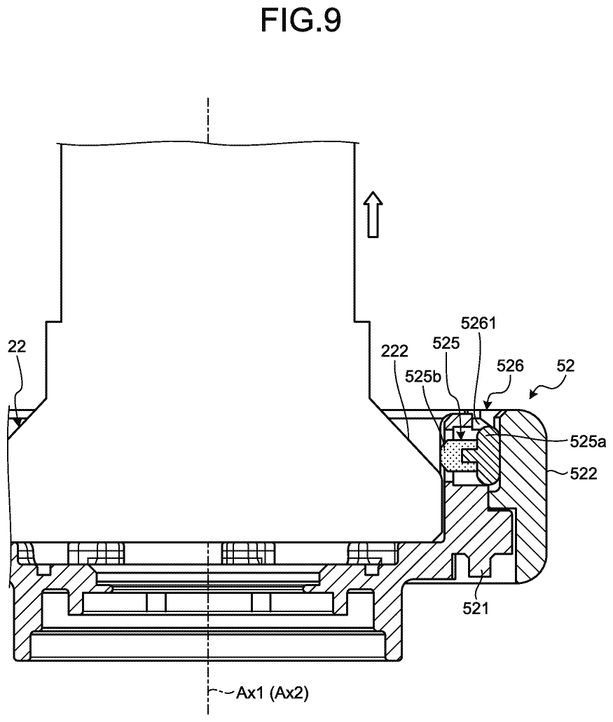

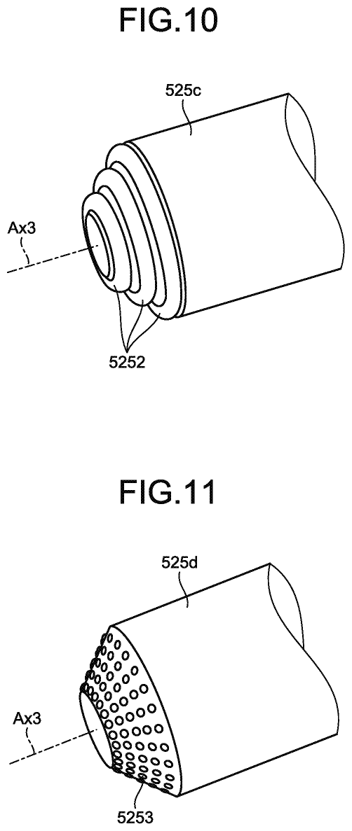

FIG. 5 is a partial sectional view illustrating the connection portion between the endoscope and the camera head;

FIG. 6 is a diagram illustrating a configuration of a locking member provided in the camera head of the endoscope apparatus in the embodiment;

FIG. 7 is a diagram illustrating a load on the locking member and an inclination portion provided in the camera head of the endoscope apparatus in the embodiment;

FIG. 8 is a diagram illustrating the connection portion between the endoscope and the camera head;

FIG. 9 is a partial sectional view illustrating the connection portion between the endoscope and the camera head;

FIG. 10 is a diagram illustrating a configuration of a locking member provided in a camera head of an endoscope apparatus according to a first modification example of the embodiment; and

FIG. 11 is a diagram illustrating a configuration of a locking member provided in a camera head of an endoscope apparatus according to a second modification example of the embodiment.

### DETAILED DESCRIPTION

Hereinafter, a form of embodying the present disclosure (hereinafter, embodiment) will be described with reference to the drawings. The present disclosure is not limited to the embodiment described below. In description for the drawings, the same parts are denoted by the same reference signs.

### Embodiment

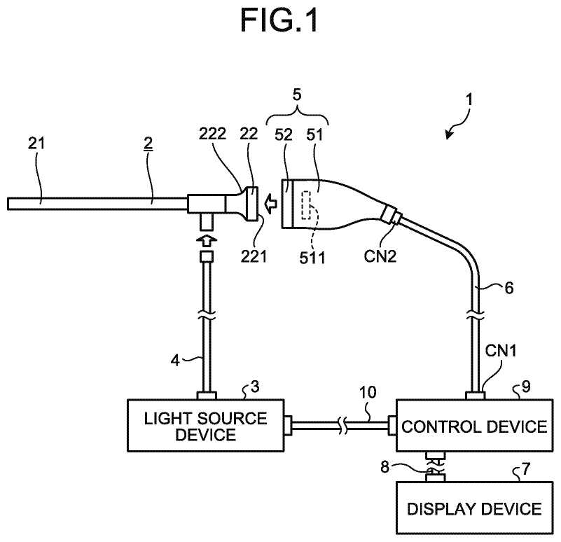

FIG. 1 is a diagram illustrating an overall configuration of an endoscope apparatus according to an embodiment. An endoscope apparatus 1 is an apparatus that is used in the medical field and observes the inside of a living body. As illustrated in FIG. 1, the endoscope apparatus 1 includes an endoscope 2, a light source device 3, a light guide 4, a camera head 5, a first transmission cable 6, a display device 7, a second transmission cable 8, a control device 9, and a third transmission cable 10.

The endoscope 2 is configured as a rigid endoscope. That is, the entirety of the endoscope 2 is rigid, or a portion of the transmission cable is flexible and other portions thereof are rigid. The endoscope 2 has an elongated shape and is inserted into a living body. As illustrated in FIG. 1, the endoscope 2 includes an insertion portion 21 and an eyepiece unit 22.

The insertion portion 21 is a portion which extends in a straight line and is inserted into the living body. An optical system (not illustrated) which is configured with one or a plurality of lenses and condenses an object image is provided in the insertion portion 21.

The eyepiece unit 22 is provided at a proximal end (right end portion in FIG. 1) of the insertion portion 21. An eyepiece optical system (not illustrated) is provided in the eyepiece unit 22 and emits the object image condensed by the optical system (not illustrated) in the insertion portion 21, from the eyepiece unit 22 to the outside of the eyepiece unit 22. The eyepiece unit 22 has a diameter increasing toward the proximal end.

One end of the light guide 4 is connected to the light source device 3, and thus the light source device 3 supplies light for lighting the inside of the living body to the one end of the light guide 4 under control of the control device 9.

The light guide 4 has the one end which is detachably connected to the light source device 3 and the other end which is detachably connected to the endoscope 2. The light guide 4 propagates light supplied from the light source device 3 from the one end to the other end, to supply the light to the endoscope 2. The light supplied to the endoscope 2 is emitted from a distal end (left end portion in FIG. 1) of the endoscope 2 and is used for irradiating the inside of the living body. Light (object image) reflected by the inside of the living body after the inside of the living body is irradiated is condensed by the optical system (not illustrated) in the insertion portion 21.

The camera head 5 includes a sealing unit 51 (FIG. 1) and a coupler member 52. In the sealing unit 51, an image pickup element 511 and the like are stored air-tightly or water-tightly. The coupler member 52 is provided in the sealing unit 51 and is detachably connected to the eyepiece unit 22 of the endoscope 2. The camera head 5 captures the object image condensed by the endoscope 2 and outputs an image signal (RAW signal) by the capturing, under control of the control device 9. The image signal is an image signal of 4K or higher, for example.

The image pickup element 511 is provided on an optical path of light guided by the optical system of the connected endoscope 2.

A specific shape of the coupler member 52 will be described later.

One end of the first transmission cable 6 is detachably connected to the control device 9 through a connector CN1, and the other end of the first transmission cable 6 is detachably connected to the camera head 5 through a connector CN2. The first transmission cable 6 transmits an image signal and the like output from the camera head 5 to the control device 9 and transmits a control signal, a synchronization signal, a clock, power, and the like output from the control device 9 to the camera head 5.

Regarding transmission of the image signal from the camera head 5 to the control device 9 through the first transmission cable 6, the image signal may be transmitted in a form of an optical signal or an electric signal. The control signal, the synchronization signal, and the clock are transmitted from the control device 9 to the camera head 5 through the first transmission cable 6 in the similar manner.

The display device 7 is configured with a display using liquid crystal, organic electro luminescence (EL), or the like. The display device 7 displays the captured image based on a video signal from the control device 9 under control of the control device 9.

One end of the second transmission cable 8 is detachably connected to the display device 7, and the other end of the second transmission cable 8 is detachably connected to the control device 9. The second transmission cable 8 transmits the video signal processed by the control device 9 to the display device 7.

The control device 9 includes a central processing unit (CPU) and collectively controls operations of the light source device 3, the camera head 5, and the display device 7.

For example, the control device 9 generates a video signal by performing various kinds of processing on the image signal acquired from the camera head 5 through the first transmission cable 6, and outputs the generated video signal to the display device 7 through the second transmission cable 8. The display device 7 displays the captured image based on the video signal. The control device 9 outputs a control signal and the like to the camera head 5 or the light source device 3 through the first transmission cable 6 or the third transmission cable 10.

One end of the third transmission cable 10 is detachably connected to the light source device 3, and the other end of the third transmission cable 10 is detachably connected to the control device 9. The third transmission cable 10 transmits a control signal from the control device 9 to the light source device 3.

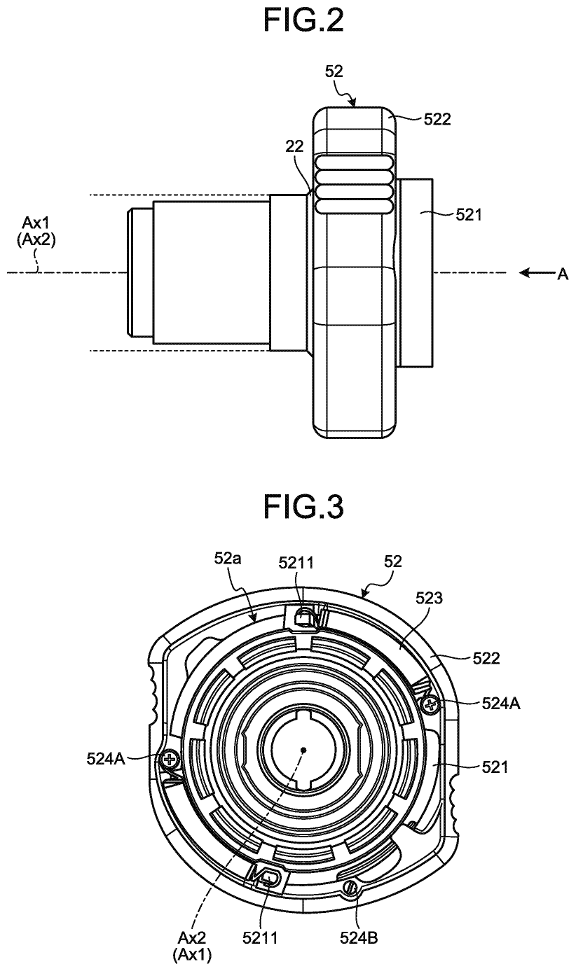

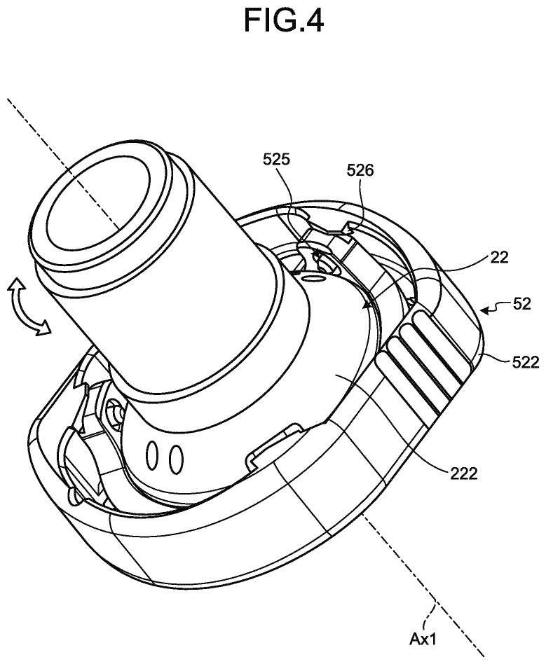

Next, the configurations of the eyepiece unit 22 and the coupler member 52 will be described. FIG. 2 is a diagram illustrating a connection portion between the endoscope 2 and the camera head 5. FIG. 3 is a diagram illustrating the configuration of the coupler member when viewed from an arrow A illustrated in FIG. 2. FIG. 4 is a diagram illustrating a connection portion between the endoscope and the camera head. FIG. 5 is a partial sectional view illustrating the connection portion between the endoscope and the camera head. It is assumed that the endoscope 2 side is a distal end side, and an opposite side of the endoscope 2 side in a direction of a central axis Ax2 is a proximal end side in the coupler member 52. Descriptions will be made on the assumption that, when the endoscope 2 is coupled to the coupler member 52, a central axis Ax1 of the endoscope 2 coincides with the central axis Ax2 of the camera head 5 (coupler member 52).

FIGS. 2 to 5 illustrate a state where the endoscope and the camera head are connected to each other, and this connection is locked. FIGS. 2 and 4 illustrate only a portion of an internal structure (jig) of the eyepiece unit 22 and the insertion portion 21 in the endoscope 2.

The eyepiece unit 22 has a substantially cylindrical shape. In the eyepiece unit 22, an end surface on the proximal end side (right end portion side in FIG. 2) extends along the entire circumference in a circumferential direction which is perpendicular to the central axis Ax1 and centers on the central axis Ax1. This end surface functions as an abutting surface 221 (see FIG. 1) according to the present disclosure.

A distal end side of an outer circumferential surface of the eyepiece unit 22 has a tapered shape having a diameter which decreases toward the distal end. Thus, an outer circumferential surface of the distal end side becomes farther from the central axis Ax1 while being directed toward the abutting surface 221, and functions as an inclination surface 222 according to the present disclosure. The inclination surface 222 corresponds to an eyepiece unit-side abutting surface.

The coupler member 52 includes a mount 521 and a ring 522. The mount 521 has a substantially columnar shape. The ring 522 is provided at an outer circumference of the mount 521 and is rotatable around the central axis Ax2. In the coupler member 52, a coupling recess portion 52a is provided on an end surface on a distal end side (left end portion side in FIG. 2). The coupling recess portion 52a is recessed toward the proximal end side (right end portion side in FIG. 2) by the mount 521 and the ring 522. The eyepiece unit 22 is inserted into the coupling recess portion 52a. In a state where the eyepiece unit 22 is coupled to the coupler member 52 by inserting the eyepiece unit 22 into the coupling recess portion 52a, the central axis Ax1 coincides with the central axis Ax2 of the coupler member 52. In a state where the eyepiece unit 22 is coupled to the coupler member 52, the endoscope 2 is rotatable around the central axis Ax1.

A spring 523 is provided in the coupler member 52. Rotation of the ring 522 with respect to the mount 521 is restricted by a biasing force of the spring 523. The spring 523 is attached to a protrusion portion 5211 formed in the mount 521 and to a screw 524A attached to the ring 522. Specifically, one end of the spring 523 is attached to the protrusion portion 5211, and the other end of the spring 523 is attached to the screw 524A. In a state where connection between the endoscope 2 and the camera head 5 is locked, as illustrated in FIGS. 2 to 5, the spring 523 is in a reduced state.

The mount 521 and the ring 522 are attached to each other by a screw 524B such that the ring 522 is not separated from the mount 521.

In the state where the connection between the endoscope 2 and the camera head 5 is locked, the endoscope 2 is not separated from the camera head 5, a locking member 525 (see FIGS. 4 and 5) provided in the mount 521. In a locking state, a distal end of the locking member 525 protrudes toward the central axis Ax1 and locks on the inclination surface 222, at the coupling recess portion 52a. In the embodiment, descriptions will be made on the assumption that three locking members 525 are provided at positions different from each other. However, at least one locking member 525 may be provided, and the number of locking members 525 may be set to any value. For example, in a case where one or two locking members 525 are provided, a protrusion that locks the eyepiece unit 22 may be provided at a position different from the position of the locking member 525. The locking member 525 may be applied to at least one of components locking the eyepiece unit 22.

A distal end of the locking member 525 penetrates the mount 521, and a proximal end of the locking member 525 abuts on an inner wall of the ring 522. A biasing member 526 that applies a biasing force for the eyepiece unit 22 to the locking member 525 is provided in the ring 522. The biasing member 526 abuts on the locking member 525 and changes the position or the direction of the locking member 525.

The thickness of the biasing member 526 on an inner circumferential side is thicker than thicknesses of other portions of the ring 522. An inclination portion 5261 inclined from the central axis Ax2 is formed on a distal end side of the biasing member 526. Specifically, the biasing member 526 has a thickness which gradually increases in a circumferential direction of the ring 522.

When the locking member 525 is in the locking state, the biasing member 526 is brought into contact with the locking member 525 and moves the locking member 525 toward the central axis Ax2. At this time, the inclination portion 5261 comes into contact with the locking member 525 and changes the direction of an end portion of the locking member 525 on which the locking member 525 comes into contact with the inclination surface 222, to the inclination surface 222 side.

FIG. 6 is a diagram illustrating a configuration of the locking member provided in the camera head of the endoscope apparatus according to the embodiment. The locking member 525 has a base portion 525a as a foundation and an extension portion 525b extending from the base portion 525a.

The base portion 525a has a curved outer surface. The base portion 525a has a protrusion to be joined to the extension portion 525b (see FIG. 5).

An inclination surface 5251 formed in the extension portion 525b. The inclination surface 5251 is formed at an end portion on an opposite side of a side on which the extension portion 525b is joined to the base portion 525a. The inclination surface 5251 has an angle of, for example, about 50° to a central axis Ax3. The extension portion 525b corresponds to an abutting portion, and the inclination surface 5251 corresponds to an abutting portion-side abutting surface.

The extension portion 525b is formed with a rigid material. Examples of the material forming the extension portion 525b include metal, an alloy, an engineering plastic (including a general purpose engineering plastic and a super engineering plastic), and ore. In addition, in order to impart chemical resistance to the extension portion 525b, SUS, titanium, or an engineering plastic having Rockwell hardness of R100 or larger is preferably used. As the engineering plastic having Rockwell hardness of R100 or larger, a super engineering plastic, for example, polyether ether ketone (PEEK) is exemplified. Further, in order to suppress scratch of the eyepiece unit 22, the material of the extension portion 525b preferably has hardness lower than hardness on the surface of the eyepiece unit 22. On the contrary, in order to more suppress wear of the extension portion 525b, the material of the extension portion 525b preferably has hardness higher than the hardness on the surface of the eyepiece unit 22.

FIG. 7 is a diagram illustrating a load on the locking member and the inclination portion provided in the camera head of the endoscope apparatus according to the embodiment. The locking member 525 is inclined by being brought into contact with the basing member 526, and thus the direction of the distal end changes (arrows Y1 and Y2 in FIG. 7). A changeable range (moving range in directions indicated by the arrows Y1 and Y2) of the direction of the distal end is equal to or wider than a tolerance range. At this time, a load from the inclination portion 5261 of the biasing member 526 is applied to the locking member 525 (corresponding to a direction of a resultant force described later). The load corresponds to the resultant force F₂ of a force F₁ in a direction (here, corresponding to the central axis Ax3) perpendicular to the optical path of light guided from the endoscope 2 and a force F₂ in a direction parallel to the optical path of the light guided to the endoscope 2.

When locking the endoscope 2 (here, inclination surface 222) as a connection destination, the locking member 525 applies a load to the inclination surface 222. A direction (indicated by an arrow Y3) of the load applied to the inclination surface 222 by the locking member 525 is substantially parallel to a direction of the load (resultant force F₃) applied to the locking member 525 by the biasing member 526. As described above, in a state where the direction of the locking member 525 is adjusted by the biasing member 526, and then is brought into contact with the inclination surface 222, the locking member 525 comes into line contact with the inclination surface 222. Surface contact may occur by elastic deformation of the extension portion 5251, The line contact (or surface contact) is obtained by a set of point contacts at a plurality of points. Since locking member 525 comes into line contact with the inclination surface 222, when the endoscope 2 rotates around the central axis Ax1, the moment (rotational torque) of a force received around the central axis Ax1 by the endoscope 2 with respect to the rotation is larger than the moment in a case of the point contact. At this time, the normal force (N(=F₀/μ:μ indicates a static friction coefficient) corresponding to the maximum static friction force (F)) in a state where the extension portion 525b is in contact with the inclination surface 222 is larger than a force which is applied to the extension portion 525b by the biasing member 526 and has the same direction as the direction of the above-described normal force. The direction of the normal force is, for example, the direction indicated by the arrow Y3 illustrated FIG. 7.

The endoscope 2 may be detached from the camera head 5 by causing the ring 522 to rotate.

FIG. 8 is a diagram illustrating the connection. portion between the endoscope and the camera head. FIG. 9 is a partial sectional view illustrating the connection portion between the endoscope and the camera head;

FIGS. 8 and 9 illustrate a state where the endoscope and the camera head are connected to each other, and the connection is unlocked. FIGS. 8 and 9 illustrate only a portion of the internal structure (jig) of the eyepiece unit 22 and the insertion portion 21 in the endoscope 2.

If the ring 522 is rotated around the central axis Axe with respect to the mount 521, an inner wall of the ring 522 including the biasing member 526 is separated from the locking member 525. Thus, the locking member 525 is movable toward the outer circumference of the coupler member 52, and thus a locking state of the locking member 525 onto the inclination surface 222 may be released.

If, in a state where the position of the ring 522 illustrated in FIGS. 8 and 9 is maintained, the eyepiece unit 22 of the endoscope 2 is attached, and then the ring 522 is caused to rotate ((be brought back) to a position illustrated in FIGS. 4 and 5, the locking member 525 comes into contact with the biasing member 526 again and moves toward the central axis Ax1. (see FIGS. 5 and 7). At this time, the inclination portion 5261 abuts on the base portion 525a, and the load (resultant force illustrated in FIG. 7 is applied to the base portion 525a. If the load (resultant force F₃) is applied to the base portion 525a, the extension portion 525b pivots to change the direction to the direction indicated by the arrow Y1 or Y2 illustrated in FIG. 7. Thus, the inclination surface 5251 abuts on the inclination surface 222, and the load in the direction in by the arrow Y3 is applied to the eyepiece unit 22.

In the above-described embodiment, the configuration in which the eyepiece unit 22 of the endoscope 2 comes into line contact with the rigid extension portion 525b, and thereby preventing removal of the endoscope 2 from the coupler member 52 while the endoscope 2 is caused to be rotatable around the central axis Ax1 is made. According to the embodiment, since the extension portion 525b is rigid, it is possible to suppress the wear of the locking member 525 (extension portion 525b) due to rotation of the endoscope 2 even though the endoscope 2 rotates (slides).

In the above-described embodiment, when the extension portion 525b is brought into line contact with the eyepiece unit 22, and the endoscope 2 rotates around the central axis Ax1, the moment (rotational torque) of the force received around the central axis Ax1 by the endoscope 2 with respect to the rotation is larger than the moment in a case of contact at one point.

For example, in a case where the locking member is brought into contact with the eyepiece unit 22 at one point, if the rotational torque when the endoscope 2 is rotated around the central axis Ax1 is set to be large, providing a spring or increasing the size of the spring which has been originally provided is required for increasing the rotational torque. In this case, the size of the coupler member 52 increases.

On the contrary, in the embodiment, the rotational torque increases by bringing the extension portion 525b into line contact with the eyepiece unit 22. Thus, the increase of the size of the coupler member 52 is suppressed even though the rotational torque increases.

In the above-described embodiment, the ring 522 is rotated, and thus the direction of the locking member 525 (extension portion 525b) is changed while controlling the locking state of the locking member 525, such that the biasing member 526 comes into line contact with the inclination surface 222 of the eyepiece unit 22 of the coupled endoscope 2. Accordingly, it is possible to also handle a case where an endoscope 2 having different standards (size or angle of the inclination surface 222) is coupled.

### First Modification Example

A first modification example of the embodiment will be described. An endoscope apparatus according to the first modification example has the same configuration as the configuration of the above-described endoscope apparatus 1 except for only the locking member provided in the coupler member 52. Components different from those in the above-described embodiment will be described below. In the first modification example, an extension portion 525c is provided instead of the extension portion 525b of the above-described locking member 525. FIG. 10 is a diagram illustrating a configuration of a locking member provided in a camera head of the endoscope apparatus according to the first modification example of the embodiment.

The extension portion 525c is formed with the same material as the material of the above-described extension portion 525b. The extension portion 525c on an opposite side of a side on which the extension portion 525c is joined to the base portion 525a (see FIG. 6) has a diameter decreasing toward a distal end. A plurality of annular projection portions 5252 is provided in the extension portion 525c. The annular projection portions 5252 are formed at an end portion (diameter-reduced portion) on the opposite side of the side on which the extension portion 525c is joined to the base portion 525a (see FIG. 6). The extension portion 525c corresponds to the abutting portion.

The annular projection portion 5252 refers to an annular protrusion. The plurality of annular projection portions 5252 is arranged along the central axis Ax3 of the locking member (extension portion 525c). Since a distal end of the extension portion 525c has a decreasing diameter, in the plurality of annular projection portions 5252, the annular projection portion 5252 closer to the distal end of the extension portion 525c on the central axis Ax3 has a smaller diameter. When the endoscope 2 is coupled, each of the annular projection portions 5252 abuts on the inclination surface 222. Thus, when the endoscope 2 rotates around a central axis Ax1, the moment (rotational torque) of a force received around the central axis Ax1 by the endoscope 2 with respect to the rotation is larger than the moment in a case of contact at one point.

### Second Modification Example

A second modification example of the embodiment will be described. An endoscope apparatus according to the second modification example has the same configuration as the configuration of the above-described endoscope apparatus 1 except for only the locking member provided in the coupler member 52. Components different from those in the above-described embodiment will be described below. In the second modification example, an extension portion 525d provided instead of the extension portion 525b of the above-described locking member 525. FIG. 11 is a diagram illustrating a configuration of a locking member provided in a camera head of the endoscope apparatus according to the second modification example of the embodiment.

The extension portion 525d is formed with the same material as the material of the above-described extension portion 525b. The extension portion 525d on an opposite side of a side on which the extension portion 525d is joined to the base portion 525a (see FIG. 6) has a diameter decreasing toward a distal end. A plurality of embossed portions 5253 is provided in the extension portion 525d. The embossed portions 5253 are formed at an end portion on the opposite side of the side on which the extension portion 525d is owned to the base portion 525a (see FIG. 6). The extension portion 525d corresponds to the abutting portion.

The embossed portion 5253 refers to a hemispherical protrusion. The plurality of embossed portions 5253 is arranged along the central axis Ax3 of the locking member (extension portion 525d) and the circumferential direction. Since the plurality of embossed portions 5253 abuts on the inclination surface 222, when the endoscope 2 rotates around the central axis Ax1, the moment (rotational torque) of a force received around the central axis Ax1 by the endoscope 2 with respect to the rotation is larger than the moment in a case of contact at one point.

Hitherto, the embodiment is described, but the present disclosure is not limited only by the above-described embodiment.

In the above-described embodiment, the descriptions are made on the assumption that the base portion 525a is separate from the extension portions 525b to 525d. However, the base portion 525a and the extension portions 525b to 525d may be integrally formed. At this time, the material used for the locking member is a rigid material as described above.

In the above-described embodiment, the configuration in which the outer surface of the base portion 525a is set to a curved surface, the inclination portion 5261 (inclination surface) is provided in the biasing member 526, and the direction of the extension portion 525b is adjusted is described as an example. However, a configuration in which the outer surface of the base portion 525a is set to an inclination surface, and a curved surface is provided in the biasing member 526 may be made.

As described above, the camera head according to the present disclosure is useful for suppressing wear of a locking member that locks an endoscope, due to rotation of the endoscope.

According to the present disclosure, an effect that it is possible to suppress wear of the locking member to lock the endoscope, due to rotation of the endoscope.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A camera head comprising:
a coupler member which is detachably connected to an eyepiece unit of an endoscope and configured to capture an object image emitted from the eyepiece unit, wherein the coupler member includes
an abutting portion which is rigid and configured to abut on the eyepiece unit at a plurality of points when at least the eyepiece unit is coupled, and
a biasing member configured to apply a biasing force for the eyepiece unit to the abutting portion.

2. The camera head according to claim 1,
wherein an eyepiece unit-side abutting surface is provided in the eyepiece unit to extend along an entire circumference of the eyepiece unit in a circumferential direction centering on a central axis of the endoscope in an insertion direction into a subject, and
the biasing member inclines the abutting portion to a side abutting on the eyepiece unit-side abutting surface.

3. The camera head according to claim 2, wherein the abutting portion comes into line contact or surface contact with the eyepiece unit-side abutting surface.

4. The camera head according to claim 2, wherein the biasing member is configured to bias a resultant force of a force in a direction perpendicular to an optical path of light guided from the endoscope and a force in a direction parallel to the optical path of the light guided from the endoscope, as the biasing force, to the abutting portion.

5. The camera head according to claim 1, wherein an abutting portion-side abutting surface extending along an entire circumference of the abutting portion in a circumferential direction centering on a central axis of a member in which the abutting portion is formed is formed in the abutting portion.

6. The camera head according to claim 1,
wherein the abutting portion includes a plurality of annular protrusions, and
the plurality of annular protrusions is arranged along a central axis of the abutting portion.

7. The camera head according to claim 1,
wherein the abutting portion includes a plurality of hemispherical protrusions, and
the plurality of hemispherical protrusions is arranged along a central axis of the abutting portion and a circumferential direction of the abutting portion.

8. The camera head according to claim 1, comprising:
a plurality of the abutting portions provided at positions different from each other.
